(19) United States
(12) Patent Application Publication
(10) Pub. No.: US 2003/0229318 A1
(43) Pub. Date: Dec. 11, 2003

(54) Device and methods for bile duct access and targeted delivery of fluid to liver and pancreas

(76) Inventor: Vladimir Subbotin, Madison, WI (US)

Correspondence Address: Mark K. Johnson, Mirus Corporation, 505 S. Rosa Rd., Madison, WI, 53719, US

(21) Appl. No.: 10/456,094

(22) Filed: Jun. 6, 2003

Related U.S. Application Data
(60) Provisional application No. 60/386,681, filed on Jun. 6, 2002.

(51) Int. Cl. A61M 25/00

(52) U.S. Cl. 604/264; 604/522; 606/205

(57) ABSTRACT

A device for bile duct access and targeted delivery of fluid to the liver and pancreas and methods of use are described. The device allows improved delivery of fluid without causing damage the bile duct or surrounding tissues.

### CROSS-REFERENCE TO RELATED APPLICATIONS This application is related to prior provisional application U.S. Serial No. 60/386,681 filed Jun. 6, 2002.

### BACKGROUND OF THE INVENTION

Liver and pancreas in situ perfusion via bile/pancreatic duct in small rodents is frequently performed for a variety of experimental terminal and chronic protocols, such as liver/pancreas gene delivery, liver/pancreas perfusion for cell/islet separation/purification, induction of liver/pancreas damage, etc. However, the perfusion via bile/pancreatic duct still constitutes a technical problem, especially when animals are intended to survive after procedure. The main technique currently used for bile/pancreatic duct canulation is a bile duct puncture with a 30 G syringe needle and manual injection with 1 ml syringe. This technique has two pitfalls: 1) poor ability to control the speed of injection, and 2) bile duct damage by a sharp needle during the initial puncture and unavoidable additional damage due to bile duct movement, such as caused by animal respiration, during the injection. The second complication results in high animal mortality in chronic experiments due to bile leakage and animal toxicity.

The following is a description of the existing techniques and their disadvantages.

1. One step trans-bile duct access with bile duct undergoing invasive manipulation (puncture) with 30G or 33 G needles and manual syringe injection [1, 2]. Either a metal microvessel clamp or a tie is placed around the bile duct to prevent back flow. This technique is associated by design with bile duct damage and typically results in bile leakage and animal death. This design permits only a manual injection with a syringe approach and requires suturing or prolonged application of Gelatin sponge.

2. Multi-step trans-bile duct access with bile duct undergoing invasive manipulation (puncture) and cannulation with a polyethylene tube followed by pump/syringe injection. After injection the tube end is kept in the bile duct and the other end was inserted into the duodenum so that bile flows into the duodenum through the tube [3]. This technique, while avoiding bile leakage from the injection site, causes bile duct damage. This technique also requires a prosthetic bile duct anastomosis connection to the duodenum. Potential post-insertion complications include duodenum inflammation and peritonitis.

3. Trans-duodenal bile duct access (mult-step with sphincterotomy). A 23-gauge needle is used to make an opening in the duodenum and to perform a sphincterotomy on the sphincter of Oddi. A polyethylene catheter is inserted through the duodenal opening and advanced in a retrograde direction through the sphincter of Oddi into the common bile duct. The catheter is advanced so that its tip can be visualized just rostral to the junction with the superior pancreatic duct. A 6-0 silk tie is placed around the common bile duct and used to secure the catheter in position [4]. Sphincterotomy on the sphincter of Oddi inevitably leads to bile drainage dysfunction. This method is technically difficult, particularly when inserting a flexible polyethylene catheter into the whole length of bile duct, past the upper pancreatic duct, when the liver is a target.

4. Trans-duodenal bile duct access (multi step with guiding nylon thread). The duodenal wall is punctured with a 26-gauge needle, and a 5-0 nylon thread is introduced as a guide through the puncture site into the papilla of Vater and sphincter of Oddi up to the hepatic duct. A polyethylene catheter is inserted over the 5-0 nylon thread up to the hepatic duct, followed by removal of the nylon thread [5]. As with technique #3 above, sphincterotomy on the sphincter of Oddi inevitably leads to bile drainage dysfunction. With this technique, extra time is required for introduction of a 5-0 nylon thread guide into the papilla of Vater, sphincter of Oddi and bile duct, and for its removal. Inserting a flexible polyethylene catheter though papilla of Vater, sphincter of Oddi and bile duct is procedurally difficult. Extra time is also required for connection of a flexible polyethylene catheter to a syringe.

### SUMMARY OF THE INVENTION

In a preferred embodiment, we describe a bile duct catheter that allows delivery of a solution to the liver or pancreas in small animals comprising: a hollow shaft containing a non-traumatic tip. The bile duct catheter is inserted into the bile duct through a small puncture of the duodenal wall and through the sphincter of Oddi. A fitted clamp may be used together with the catheter to restrict the direction of fluid flow through the bile duct. The solution may contain a biologically active compound such as a polynucleotide, a drug or a cell. The bile duct catheter may be used to deliver the biologically active compound to a liver cell, a bile duct epithelial cell, a pancreas cell, or a pancreatic exocrine cell. The solution may also be delivered to perfuse the liver, pancreas or bile duct.

Further objects, features, and advantages of the invention will be apparent from the following detailed description when taken in conjunction with the accompanying drawings.

### BRIEF DESCRIPTION OF THE FIGURES

FIG. 1. Photograph of the bile duct catheter showing the shaft (1), tip (2), plastic handle (3), shaft extension (4) and a microvessel clamp (5).

FIG. 2. Photograph of the bile duct catheter tip (2) and a standard syringe needle tip (1).

FIG. 3. Illustration of a cross-section of the bile duct catheter (1) inside of a bile duct (3). Also shown are a standard microvessel clamp (2) and the luminal space generated between the catheter shaft and the bile duct (4) formed using the standard microvessel clamp.

FIG. 4. Illustration of a crossection of the bile duct catheter (1) inside of a bile duct (3) with the specialized clamp. The specialized clamp contains pliable grips (5) attached to the metal sides (2) of a standard microvessel clamp. The pliable grip provide for improve occlusion of the bile duct (3) around the catheter shaft (1). A channel is present in one or both grips. The illustration shows channels present in both grips.

FIG. 5. Photograph of the specialized clamp (1) with pliable grips (2) with a channel (3) present in one of the grips.

FIG. 6. Image of DNA/PEI/PAA complexes delivered to bile duct epithelial cells using the bile duct catheter. Nuclei and actin are shown in gray, DNA particles are shown in white. BD—bile duct lumen, BD branch—bile duct branches;>—bile canaliculae; PV—portal vein, HA—hepatic artery.

FIG. 7. Expression of β-galactosidase following delivery of DNA/PEI/PAA complexes delivered to bile duct epithelial cells following bile duct occlusion using the bile duct catheter. A. and B. 100× magnification. C. 200× magnification. D. 400× magnification.

FIG. 8. Successful transfections of bile ducts following delivery of DNA complexes using the bile duct catheter performed on a background of biliary-associated liver cirrhosis.

FIG. 9. Hepatoma formation in liver of C57BL mouse following delivery of Hepa1-6 cells using the bile duct delivery device.

FIG. 10. colonic liver neoplasms in liver of C57BL mouse following delivery of MC38 cells using the bile duct delivery device.

FIG. 11. Gene delivery to pancreatic ducts and exocrine cells in ICR mouse. β-galactosidase expression following delivery of pCILacZ into the distal bile duct with proximal bile duct clamped at bifurcation.

### DETAILED DESCRIPTION

To avoid bile duct damage and better control speed of injection we have invented a new trans-intestinal method and device comprising: puncture of the duodenal wall in the vicinity of the sphincter Oddi with a 27G needle and advancing specially designed bile duct catheter into the duodenum lumen, sphincter of Oddi, and bile duct. A specialize clamp further limits direction of injected fluid flow through the bile duct after the catheter is inserted. Puncture of the duodenal wall with a needle allows the insertion of a blunt-ended catheter. The blunt-ended catheter can then be advanced though the sphincter Oddi and bile duct without causing damage to either tissue. Previous methods and devices for delivering a solution to the bile duct have caused damage to tissue as described above.

The bile duct catheter comprises: a hollow shaft of at least 30 mm in length. The shaft may be straight or slightly curved. At one terminus of the shaft is a non-traumatic tip. The hollow cavity of the shaft or cylinder extends from the non-traumatic tip end through the shaft to the opposite, external end of the shaft. Fluid inserted into the cavity at the external end of the shaft exits the shaft into the animal at the non-traumatic tip end of the shaft. The shaft may be made of any non-porous material suitable for a surgical instrument that allows the catheter to be inserted into the bile duct without damaging the duct. The material must be sufficiently rigid to allow guidance of the shaft through the duct. Suitable materials include, but are not limited to, polished metals. The outside diameter of the shaft must be small enough to enter though the sphincter Oddi and into lumen of the duct without causing damage to the sphincter Oddi or the duct. An outside diameter of the shaft that is near to the diameter of the duct is preferred. This diameter allows the duct to be more readily clamped in such a way that fluid movement between the outside walls of the shaft and the inside walls of the duct may be occluded. The inside diameter of the shaft must be large enough to allow fluid to be injected though the shaft at a sufficient rate for the given usage. In addition to the portals at either end of the shaft, the shaft may also have a handle. The handle is located approximately 10-60 mm from the non-traumatic tip end of the shaft and is affixed to the outside to the shaft. Preferably, the handle is located 30-40 mm from the non-traumatic tip end of the shaft. The handle may be of any size as long as it does not interfere with the function of the catheter. The handle may be made of any material suitable for a surgical instrument. The handle allows for improved manual manipulation of the catheter. The handle may be removable. One or more portals at the external end of the catheter are designed to allow attachment, such as by flexible tubes, to one or more containers. Fluid may be inserted through the flexible tube and enter the cavity of the catheter shaft through the external end of the shaft. The fluid is then delivered to the animal through the catheter shaft. The container holds the fluid to be injected through the catheter into the animal.

The non-traumatic tip of the catheter shaft may seem to resemble a syringe needle tip. In contrast to a traumatic syringe needle tip, the non-traumatic tip does not have either a sharply pointed tip or a cutting edge. The non-traumatic tip has a shallower bevel than a standard syringe needle end. From the line of the longitudinal length of the shaft, the angle of the bevel is larger for the non-traumatic tip than for a typical syringe end. 90° would indicate no bevel with decreasing angles going from <90° to >0°. Alternatively, the shaft can have non-traumatic tip that consists of rounded, oval, or half-elliptical terminus. The requirement is that catheter shaft be able to be guided through the lumen of the bile duct without cutting or otherwise damaging the duct or other tissue. The portal at the end of the catheter shaft may either proceed directly though the terminus of the cylinder, as with a syringe needle. One or more portals may be present in the side of the catheter shaft barrel. These portals may in place of or in addition to a portal through the shaft terminus.

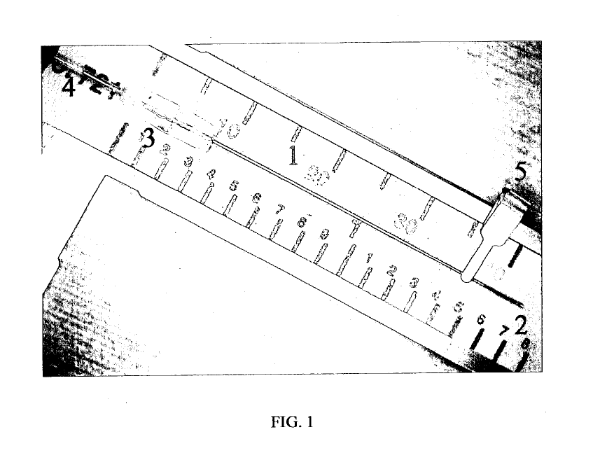

The bile duct catheter, shown in FIG. 1, comprises: 1—a hollow catheter shaft; 2— a specially shaped and polished catheter tip; 3— a handle; and, 4— a section of catheter shaft extending from plastic handle that is connected by plastic tubing to a syringe.

The catheter shaft made be made of any material that allows the catheter to be inserted into a bile duct and advanced through the bile duct without damaging the duct. A preferred material is polished stainless steel. Referring to FIG. 1, the stainless steel catheter is built having a length of the shaft (1) that can be inserted approximately 35 mm. The length is measured from the handle to the tip. This length is sufficiently long to use a trans-intestinal approach and advance the catheter into the bile duct past the merging of the proximal pancreatic duct. The hollow nature of the shaft allows fluid to be inserted from a container outside the animal though the catheter into the animal. The diameter of the shaft must small enough that the shaft can be inserted into the bile duct without causing damage to the bile duct. The shaft must be large enough to allow fluid to be inserted through the shaft at a sufficient rate.

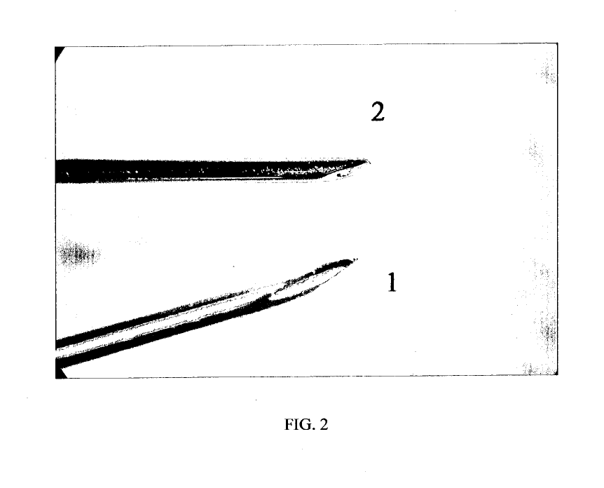

The shaped and polished tip of the catheter is capable of advancing inside the ampulla of Vater and opening the sphincter of Oddi without causing damage to intestinal or bile duct tissues. Furthermore, this non-traumatic tip allows the catheter to advance selectively, if desired, into either the right or the left bile ducts or into the gall bladder without causing damage to the aforementioned structures. Referring to FIG. 2, to avoid damage to the sphincter of Oddi and bile duct during catheterization and infusion, the tip of the catheter (2) is specially shaped and polished. The catheter has a blunted tip with edges that are not sharp or cutting. A conventional syringe needle tip (1) having a sharp point and cutting edges is shown alongside the catheter tip. In order to move the catheter inside the bile duct without causing damage, the outside diameter of the catheter must be smaller than internal diameter of bile duct lumen.

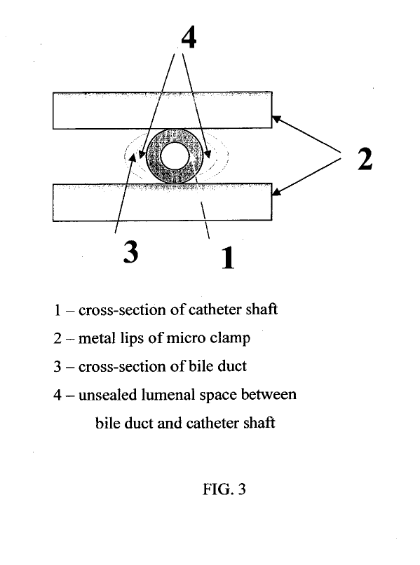

For some fluid injection protocols, the resultant positive fluid pressure can result in unwanted fluid backflow between catheter shaft and bile duct. The application of currently available micro clamps fails to prevent this unwanted fluid backflow. Referring to FIG. 3, the flat metal sides of a standard clamp (2) press against the bile duct over the metal catheter tube (1) leaving unsealed luminal space (4) between the catheter (1) and the vessel wall (3) through which fluid can flow. Therefore, specially designed clamps were designed to provide better occlusion of the vessel.

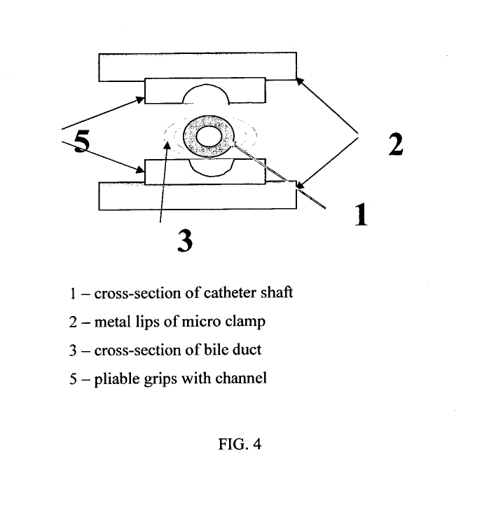

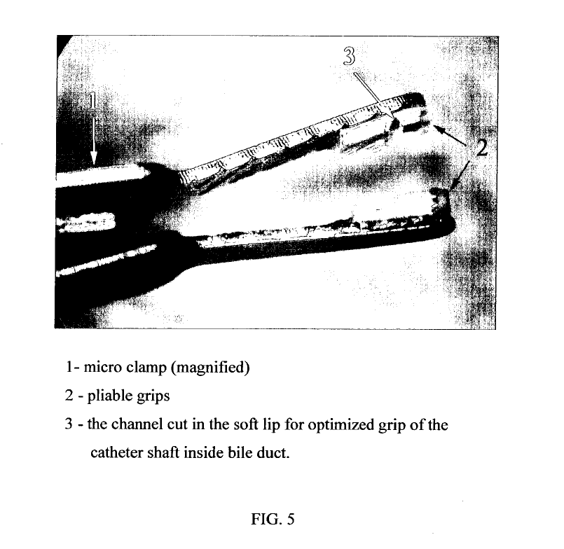

Referring to FIG. 4 and FIG. 5, the new micro clamp contains specially designed pliable grips. A channel is present in one or both of the grips for fitting to the catheter/bile duct. The pliable grips must be firm enough to provide clamping pressure, and flexible enough to provide a good seal around the catheter and duct without damaging the duct.

With this design and technique the catheter was capable to expand the sphincter of Oddi without causing damage and advance inside the bile duct toward the liver, also without causing damage to bile duct. Avoiding damage to the sphincter Oddi is crucial for chronic experiments.

The sphincter Oddi is a muscular structure consisting of smooth muscle fibers surrounding the distal part of common bile duct and the ampulla of Vater, situating in the wall of the duodenum. The sphincter Oddi regulates the passage of bile and pancreatic fluid into the duodenum. Smooth fibers around the sphincter Oddi contract to close the sphincter [6-8]. Any mechanical damage of sphincter Oddi resulted in its contraction and prolonged cholestasis [9].

The new catheter tip design made it possible to keep the catheter inside the duct for up to 30 minutes. Despite movement of the bile duct relative to the tip of the catheter due to animal respiration no damage was caused either to the bile duct or to the sphincter Oddi. The new clamp design fully prevented unwanted fluid backflow and also allowed high speed/volume delivery of solutions to liver.

In 6 week chronic experiments, animals showed 100% survival with no bile duct/liver complication for the duration of the experiment.

Insertion of the described catheter into the bile duct and securing it with the described clamp before the distal pancreatic duct, combined with clamping the bile duct between the bile duct bifurcation and the proximal pancreatic duct, provides an ideal condition for pancreas perfusion/gene delivery.

Using the described catheter and clamp, with the described technique, enables: polynucleotide transfer to bile duct epithelial cells with polynucleotide/charge polymer delivery systems, polynucleotide transfer to liver cells with high speed/volume delivery systems, polynucleotide delivery to pancreatic ducts and exocrine cells, inoculation of tumor cells into liver or pancreas, liver perfusion, pancreas perfusion for islet purification, and modeling of liver and bile duct diseases. Compare to existing techniques of administration via portal vein, the described approach is not associated with bleeding. Bile duct delivery of tumor cells allow more cells to be inoculated, therefore generating more liver metastases.

A biologically active compound is a compound having the potential to react with biological components. More particularly, biologically active compounds utilized in this specification are designed to change the natural processes associated with a living cell, tissue or organism. For purposes of this specification, a natural process is a process that is associated with a cell, tissue or organism before delivery of a biologically active compound. Biologically active compounds may be selected from the group comprising: pharmaceuticals, proteins, peptides, polypeptides, hormones, cytokines, antigens, viruses, oligonucleotides, nucleic acids, cells, tumor cells, and transfected or transformed cells.

The term polynucleotide, or nucleic acid or polynucleic acid, is a term of art that refers to a polymer containing at least two nucleotides. Nucleotides are the monomeric units of polynucleotide polymers. Polynucleotides with less than 120 monomeric units are often called oligonucleotides. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. The term polynucleotide includes deoxyribonucleic acid (DNA) and ribonucleic acid (RNA) and combinations on DNA, RNA and other natural and synthetic nucleotides.

DNA may be in form of cDNA, in vitro polymerized DNA, plasmid DNA, parts of a plasmid DNA, genetic material derived from a virus, linear DNA, vectors (P1, PAC, BAC, YAC, artificial chromosomes), expression cassettes, chimeric sequences, recombinant DNA, chromosomal DNA, an oligonucleotide, anti-sense DNA, or derivatives of these groups. RNA may be in the form of oligonucleotide RNA, tRNA (transfer RNA), snRNA (small nuclear RNA), rRNA (ribosomal RNA), mRNA (messenger RNA), in vitro polymerized RNA, recombinant RNA, chimeric sequences, anti-sense RNA, siRNA (small interfering RNA), ribozymes, or derivatives of these groups. An anti-sense polynucleotide is a polynucleotide that interferes with the function of DNA and/or RNA. Antisense polynucleotides include, but are not limited to: morpholinos, 2′-O-methyl polynucleotides, DNA, RNA and the like. SiRNA comprises a double stranded structure typically containing 15-50 base pairs and preferably 21-25 base pairs and having a nucleotide sequence identical or nearly identical to an expressed target gene or RNA within the cell. Interference may result in suppression of expression. The polynucleotide can be a sequence whose presence or expression in a cell alters the expression or function of cellular genes or RNA. In addition, DNA and RNA may be single, double, triple, or quadruple stranded. Double, triple, and quadruple stranded polynucleotide may contain both RNA and DNA or other combinations of natural and/or synthetic nucleic acids.

A pharmaceutically acceptable solution is a solution which is not biologically or otherwise undesirable for injection into a mammal, such as normal saline or Ringer's solution. The solution may be isotonic, hypotonic or weekly hypertonic. The solution may have low ionic strength such as is provided by a sucrose or glucose solution.

### EXAMPLES

1. Delivery to Bile Duct Epithelial Cells with DNA/Charge Polymers Delivery System.

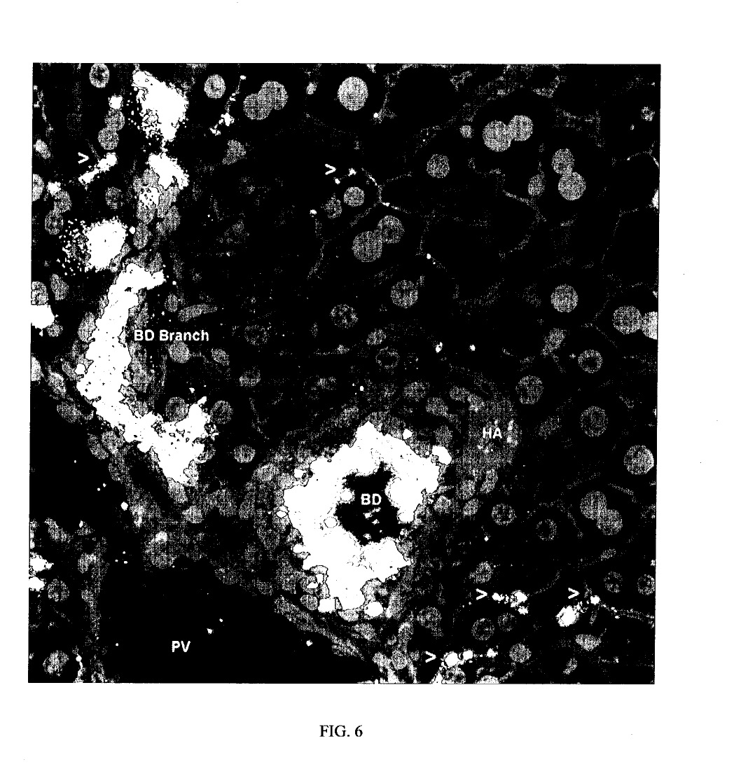

FIG. 6: pDNA/PEI/PAA particles (1:6:1) were delivered via the bile duct into ICR mice using the describe bile duct catheter. The mice were sacrificed two minutes later, the liver extracted and sections prepared. Liver sections were stained with the actin stain phalloidin-Alexa 488 (gray) and the nuclear stain To-Pro3 (gray) and examined using confocal microscopy (Zeiss LSM 510). Almost all Cy3-labeled DNA (white) appeared in the bile duct lumen (BD) and bile duct branches (BD branch), with some localization in bile canaliculae (white arrows). No pDNA signal was detected in the sinusoidal space. PV: portal vein, HA: hepatic artery.

Gene transfer with DNA/charge polymers delivery system. Successful gene transfer was performned with pDNA/PEI/PAA and LT-1 systems. We showed expression LacZ, YFP, and GFP plasmids All example given with LacZ expression. Below are example of β-Galactosidase expression in normal liver.

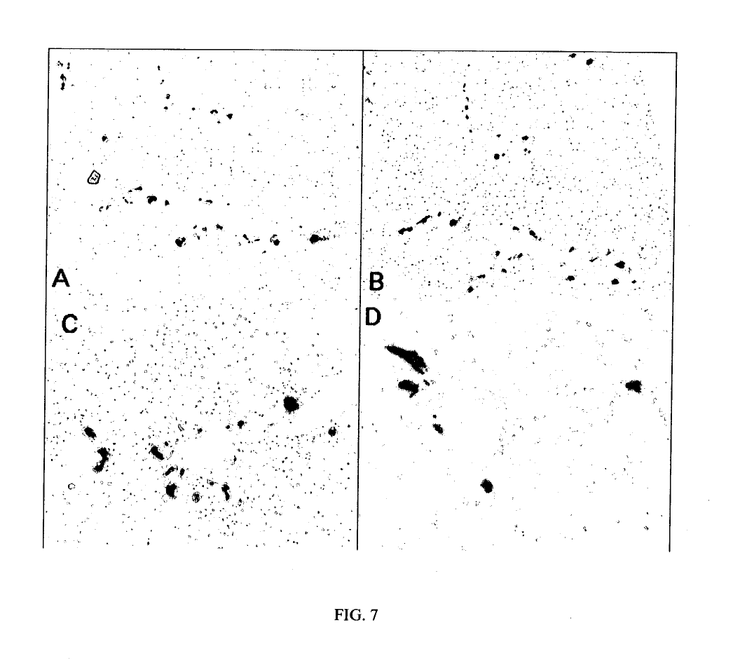

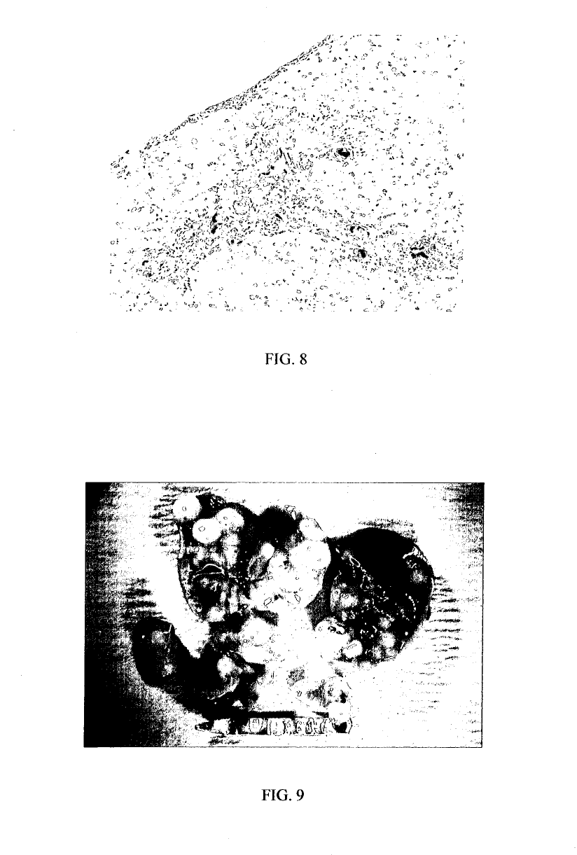

FIG. 7: Normal mouse liver. A & B sections of a liver collected 24 hours after bile duct transfection with pDNA/PEI/PAA. The two sections are 28 microns apart, stained for β-galactosidase reporter gene expression and counterstained with hematoxylin, magnification 100×. C—magnification 200×. D—location of positive cells corresponds to bile duct structure, magnification 400×.

Successful transfections of bile ducts were also performed on a background of biliary-associated liver cirrhosis.

FIG. 9. Frozen liver section of C57BL mouse 5.5 weeks after bile duct obstruction followed pCILacZ using DNA/PEI/pAA delivery system. The section was stained with X-gal staining solution (Mirus) and counterstained with hematoxylin, 200×. In all livers the β-galactosidase expressing cells we found inside fibrotic/cirrhotic tissues, and morphologically were associated with bile ducts.

Formaldehyde-fixed and paraffin embedded liver sections were used to identify the type(s) of transfected cells. Double immunostaining showed that most of β-galactosidase expressing cells were positive for pan-cytokeratin 19, the BEC marker, a proof that they are biliary epithelial cells.

2. Inoculation of Tumor Cells into Liver Using the Described Bile Duct Catheter.

The biggest advantage of bile duct delivery of tumor cells is that the number of liver metastases is dose-dependant of number of cells inoculated and metastases do not appear outside the liver. Another advantage of this approach that is not associated with bleeding, as compare with existed technique via portal vein. Successful experiments were perform with bile duct delivery of Hepa1-6 mouse hepatoma cells, and MC38 mouse colon carcinoma cells. In both tumor cell types no organs outside liver were affected.

FIG. 9. C57BL mouse. 1.5×10⁶ Hepa1-6 cells were inoculated into bile duct in 0.75 ml PBS over 1 minute. Animals were kept for 17 days. All hepatoma foci were evenly distributed between lobes in proportion to lobe mass.

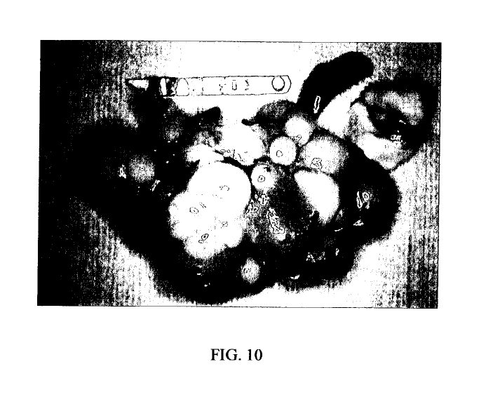

FIG. 10. C57BL mouse. 0.5×10⁵ MC38 cells were inoculated into bile duct in 0.75 ml PBS over 1 minute. Animals were kept for 14 days. All colonic liver neoplasms were evenly distributed between lobes in proportion to lobe mass. Note, there is no metastasis neither in lung nor in spleen.

4. Gene Delivery to Pancreatic Ducts and Exocrine Cells Using the Described Bile Duct Catheter.

The experiments were conducted with TransIT-LT1 and DNA/PEI/pAA using pCILacZ expression plasmid.

FIG. 11. ICR mouse. 25 μg of pCILacZ were inoculated into distal bile duct with proximal bile duct clamped at bifurcation. The section was stained with x-gal staining solution (Mirus) and counterstained with hematoxylin, 200×.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

References

1. Zhang, G., et al., Expression of naked plasmid DNA injected into the afferent and efferent vessels of rodent and dog livers. Hum Gene Ther, 1997. 8(15): p. 1763-72.

2. Zhang, X., et al., In vivo gene delivery via portal vein and bile duct to individual lobes of the rat liver using a polylysine-based nonviral DNA vector in combination with chloroquine. Hum Gene Ther, 2001. 12(18): p. 2179-90.

3. Otsuka, M., et al., In vivo liver-directed gene transfer in rats and pigs with large anionic multilamellar liposomes: routes of administration and effects of surgical manipulations on transfection efficiency. J Drug Target, 2000. 8(4): p. 267-79.

4. Wiener, S.M., et al., Manometric changes during retrograde biliary infusion in mice. Am J Physiol Gastrointest Liver Physiol, 2000. 279(1): p. G49-66.

5. Uehara, T., et al., Gene transfer to the rat biliary tract with the HVJ-cationic liposome method. J Hepatol, 1999. 30(5): p. 836-42.

6. Tzovaras, G. and B. J. Rowlands, Diagnosis and treatment of sphincter of Oddi dysfunction. Br J Surg, 1998. 85(5): p. 588-95.

7. Becker, J. M., Physiology of motor function of the sphincter of Oddi. Surg Clin North Am, 1993. 73(6): p. 1291-309.

8. Funch-Jensen, P. and N. Ebbehoj, Sphincter of Oddi motility. Scand J Gastroenterol Suppl, 1996.216: p. 46-51.

9. Hong, S. M., et al., Smooth muscle distribution in the extrahepatic bile duct: histologic and immunohistochemical studies of 122 cases. Am J Surg Pathol, 2000. 24(5): p. 660-7.

We claim:

1. A device sized to fit into a bile duct for delivering a solution to a tissue connected to a bile duct comprising: a hollow shaft with a non-traumatic tip.

2. A process for delivering a solution to a tissue connected to a bile duct, comprising:
the device of claim 1 wherein the solution consists of a biologically active compound in a pharmaceutically acceptable solution.

3. The process of claim 2 wherein the biologically active compound consists of a polynucleotide.

4. The process of claim 2 wherein the tissue consists of a liver.

5. The process of claim 1 wherein the tissue consists of a pancreas.

6. The process of claim 5 wherein the pancreas consists of exocrine cells.

7. The process of claim 2 wherein the tissue consists of a bile duct.

8. The process of claim 7 wherein the bile duct consists of bile duct epithelial cells.

9. The process of claim 3 wherein the biologically active compound consists of a cell.

10. The process of claim 1 wherein the solution is delivered to perfuse the tissue.

11. A method for delivering a solution to a tissue connected to a bile duct comprising:
inserting the device of claim 1 into the bile duct and injecting the solution though the device.

12. The method of claim 11 wherein the solution consists of a biologically active compound in a pharmaceutically acceptable solution.

13. The method of claim 12 wherein the biologically active compound consists of a polynucleotide.

14. The method of claim 11 wherein delivering the solution consists of perfusing the tissue.

15. The method of claim 14 wherein perfusing the tissue consists of isolating cells.

16. The method of claim 15 wherein the cells consist of a pancreatic islet cells.

17. The method of claim 11 wherein delivering a solution consists of modeling disease.

18. A microvessel clamp wherein pliable grips on the clamp provide for occlusion of fluid flow within the lumen of a vessel between the vessel wall and the device of claim 1.
